(19) United States
(12) Patent Application Publication
(10) Pub. No.: US 2016/0250683 A1
(43) Pub. Date: Sep. 1, 2016

(54) SECONDARY CAST ALUMINUM ALLOY FOR STRUCTURAL APPLICATIONS

(71) Applicant: GM Global Technology Operations LLC, Detroit, MI (US)

(72) Inventors: Qigui Wang, Rochester Hills, MI (US); Jason R. Traub, Clinton Township, MI (US); Michael J. Walker, Shelby Township, MI (US); Anil K. Sachdev, Rochester Hills, MI (US)

(21) Appl. No.: 14/632,308

(22) Filed: Feb. 26, 2015

(51) Int. Cl. B22D 21/00; B22D 25/02; G01N 33/20; C22C 21/02

(52) U.S. Cl. CPC B22D 21/007; C22C 21/02; B22D 25/02; G01N 33/20

(57) ABSTRACT

An aluminum alloy that can be cast into structural components wherein at least some of the raw materials used to produce the alloy are sourced from secondary production sources. In addition to aluminum as the primary constituent, such an alloy includes 5 to 14% silicon, 0 to 1.5% copper, 0.2 to 0.55% magnesium, 0.2 to 1.2% iron, 0.1 to 0.6% manganese, 0 to 0.5% nickel, 0 to 0.8% zinc, 0 to 0.2% of other trace elements selected from the group consisting essentially of titanium, zirconium, vanadium, molybdenum and cobalt. In a preferred form, most of the aluminum is from a secondary production source. Methods of analyzing a secondary production aluminum alloy to determine its constituent makeup is also disclosed, as is a method of adjusting the constituent makeup of such an alloy in situations where the alloy is out of tolerance when measured against its primary source counterpart.

The present invention relates to a heat-treatable secondary aluminum alloy that has improved casting quality and mechanical properties to facilitate casting the alloy into machinable articles such as engine blocks, cylinder heads, and transmission components for automotive and other industrial applications that takes advantage of controllable mechanical properties within such alloys.

### BACKGROUND OF THE INVENTION

The most commonly used cast aluminum alloys in structural applications in automotive and other industries include, but are not limited to, Al—Si family alloys, such as the 200 and 300 series aluminum alloys, where the inclusion of silicon (Si) is predominantly for improved castability and machinability. At least a few popular aluminum alloys (i.e., 319, 354, and 380) that are particularly useful for forming engine blocks and cylinder heads suffers from an inherent shrinkage porosity problem, mainly due to the presence of trace contaminants or alloying constituents such as strength-enhancing copper (Cu), magnesium (Mg) or manganese (Mn), among others. Known methods for heat treatment in general and solution heat treating in particular are not capable of dissolving all of the Cu in existing commercial alloys such as 319 and 380 for the subsequent age strengthening steps. This problem—which is significant in primary aluminum alloys—is exacerbated when the feedstock is a secondary aluminum (also referred to herein as “secondary production”, “secondary alloy” or the like) made from recycled or reclaimed raw material, such as aluminum cans, aircraft, automobiles, municipal waste, razed buildings or the like, where the source material for many of these reclaimed goods often comprises a mixture of many different kinds of aluminum alloys, each with varying amounts of Cu, Mn, Mg and other metals (such as zinc (Zn) or iron (Fe), among others). Of these, the presence of elevated Fe and other tramp materials can be particularly problematic for their tendency to form complex intermetallics that reduce alloy feeding capability and decrease alloy ductility, as well as lower corrosion resistance. For example, although trace amounts of Fe may be included in primary alloys in an amount of up to about 0.2 wt % (either inherently or by design as a way to help avoid die sticking or soldering), larger amounts taken from secondary production feedstock may contaminate the alloy such that a component made from such alloy falls short of thermal, mechanical or related component design requirements.

Accordingly, it is difficult or expensive to segregate secondary aluminum alloy sources to ensure a reasonable degree of constituent material homogeneity or predicability. Concomitantly, it is difficult for a designer of a complex component such as an engine block or cylinder head to work with such a material. Even if the precise proportion of the constituent ingredients is known to the designer, the presence of elevated quantities of the above constituents may make it hard to perform secondary operations (such as heat treating, additional alloying or the like) on the component being cast as a way to achieve desirable mechanical properties and low residual stresses in the final cast component.

Moreover, the use of post-casting operations may depend on the type of casting process being used. For example, solution heat treating (with its use of relatively high post-casting temperatures) may be difficult to reconcile with high pressure die casting (HPDC, also referred to as pressure die casting or more simply, die casting) due to the formation of blistering from entrapped air that is inherent in HPDC operations. Likewise, certain investment, sand or gravity castings may experience challenges in achieving high quality with commercially available secondary aluminum alloys such as 319 or 354 because of high shrinkage tendency of those secondary aluminum alloys, and particularly because of the very slow solidification rate during the casting process. Because the use of casting is often not economically viable absent some form of high-volume production techniques employing either permanent (for example, metal) or expendable (for example, lost form) molds, any use of secondary production raw material must also be compatible with the secondary operations that may be needed.

Despite these difficulties associated with using secondary production aluminum-based materials, their use in large-scale production activities (such as those associated with automotive components in general and engine blocks and cylinder heads in particular) may be justified based on the significantly lower raw material cost for recycled aluminum relative to those from comparable primary production material sources. In fact, cost concerns, as well as the desire to minimize depletion of natural resources and take advantage of significant presently-available aluminum recycling infrastructure, may lead automotive manufacturers and other large-scale users to pursue the use of secondary production components based on these alloys. To that end, there is a need of an improved castable secondary aluminum alloy that is suitable for both sand and metal mold casting and can produce high quality castings (with reduced porosity) with possibly improved alloy strength for structural applications. There is also a need for a way to determine the makeup of the secondary alloy, including accurate determination of the presence of contaminants, proper alloy constituents or the like within the alloy being contemplated for such a casting operation.

### SUMMARY OF THE INVENTION

According to one aspect of the present invention, an aluminum alloy made at least partially from secondary production aluminum is disclosed. The alloy may contain at least one of the castability and strength enhancement elements such as Si, Cu, Mg, Mn, Fe, Zn and nickel (Ni). The microstructure of the alloy consists of one or more insoluble solidified and/or precipitated particles with at least one alloying element. In one form, the alloy may include by weight approximately 5 to 14% Si, 0 to 1.5% Cu, approximately 0.2 to 0.55% Mg, 0.2-1.2% Fe, 0.1 to 0.6% Mn, 0 to 0.5% Ni, 0 to 0.8% Zn and 0 to 0.2% other trace elements such as titanium (Ti), zirconium (Zr), vanadium (V), molybdenum (Mo) and cobalt (Co), as well as a balance of aluminum.

The alloy raw material ingredient composition ranges may also be adjusted based on performance requirements of the end-use component being made from the alloy. For example, applications requiring high ductility and/or high fatigue strength may include by weight approximately 5 to 8% Si, 0 to 1.0% Cu, 0.2 to 0.4% Mg, no more than about 0.4% Fe, 0 to 0.2% Mn, 0 to 0.2% Ni, and 0 to 0.3% Zn along with the previous trace elements. Examples of components that may need such a high ductility/high fatigue strength include cylinder heads, suspension parts, aluminum wheels and shock towers. Likewise, for high tensile strength applications, the alloy may include by weight approximately 8 to 14% Si, 1.0 to 1.5% Cu, 0.4 to 0.55% Mg, no more than about 0.8% Fe, 0 to 0.3% Mn, 0 to 0.5% Ni and 0 to 0.5% Zn along with the aforementioned trace elements. Representative automobile components that may need the high tensile strength alloy may include engine blocks, engine bed plates, high pressure oil pump, control arms or the like. Moreover, for castings (in particular, high pressure die castings (HPDC)) subjected to only the precipitation (artificial aging) T5 aging process, the Cu and Mg content should be kept low, preferably below about 0.5% for Cu and about 0.2% for Mg. Components that may be made from HDPC or related operations where solution heat treating may not be used include engine blocks, transmission cases, engine covers, oil pans, transmission clutch houses or the like. In another form, since controlled solidification and heat treatment improves microstructural uniformity and refinement and provides the optimum structure and properties for the specific casting conditions, the alloy may be modified using strontium (Sr) with a preferable content of less than 0.015% by weight, and may be further grain-refined with either boron (B) or the aforementioned Ti with respective concentrations of about 0.005% by weight or about 0.15% by weight, respectively.

According to another aspect of the present invention, a method of forming a cast automotive component is disclosed. The method includes heating (for example, in a furnace) a quantity of raw materials to an amount sufficient to form an object by casting it in a mold, after which it is cooled until it solidifies into a shape defined by the mold. The material includes at least some secondary production aluminum, and may include other secondary production precursor ingredients as well. The molten material is made up of (by weight) approximately 5 to 14% silicon, 0 to 1.5% copper, 0.2 to 0.55% magnesium, 0.2 to 1.2% iron, 0.1 to 0.6% manganese, 0 to 0.5% nickel, 0 to 0.8% zinc, 0 to 0.2% of other trace elements selected from the group consisting essentially of titanium, zirconium, vanadium, molybdenum and cobalt, and the balance aluminum. In one preferred form, the raw material that is melted can be overheated (for example, up to 1000° C. for 15 to 30 minutes); this may help to completely destroy atomic cluster and heredity in the metal melt. In this way, the effects of the recycled metal that is the heart of the secondary production aluminum that can bring all kinds of element and phase segregation in the liquid metal is counteracted. For example, as the secondary aluminum alloys are usually reproduced from the recycled aluminum scraps, overheating is needed to destroy all previous history of those aluminum scraps when the secondary alloy is first reproduced. The advantage of overheating is not only to make the alloy element uniform in the materials but also to make sure that no heredity info or signature of old material remains in the newly produced alloy. Thus, reheating reduces the likelihood of having a higher volume fraction of one or more phases in the microstructure, as well as reduces the incidence of microstructure non-uniformity, even in situations where the overall alloy composition still meets the alloy specification.

According to yet another aspect of the present invention, a method of verifying the casting quality of an aluminum alloy is disclosed. As mentioned above, an elevated Fe level in an aluminum alloy is often hard to avoid when the raw materials used to make the alloy come from recycling and related secondary sources. As such, it is important to be able to determine when Fe amounts greater than about 0.2 wt % are present such that corrective measures may be taken before creating castings from such secondary aluminum alloys. One such corrective measure according to the method is to add adjustment stock such as primary recycle alloys or pre-made master alloys (typically in the form of simple binary alloy ingots such as Al-50% Si, Al-50% Mg, Al-50% Cu or the like). Such corrective measures may be undertaken for similar contaminants based on the verification discussed herein. In one form, the method includes receiving a sample of a secondary production aluminum alloy, and then generating a microstructure image corresponding to a location of interest in the sample, then measuring one or more indicia within the image so that such indicia (such as Fe intermetallic phase volume fraction) may be correlated with the presence of at least alloy constituent or at least one contaminant within the alloy. In one form, traditional chemistry analysis using inductively coupled plasma (ICP, which is also called inductively coupled plasma mass spectrometry, ICP-MS) may be used. Likewise, metallographic techniques, including those using an image analysis (IA) system, which is typically used for microstructure (phases) observation, may be employed to help ascertain the presence of alloy elements, trace elements, contaminants or the like. Another alloy or phase composition analysis method that may be used is called energy-dispersive X-ray spectroscopy (EDX) equipped in scanning electron microscopy (SEM), where a beam of electrons, protons or X-rays, excites the electron of the material being analyzed, thereby stimulating the emission of X-rays when electrons within the material are displaced. The emitted X-rays can then be measured by an energy-dispersive spectrometer as a way to measure and correlate the atomic structure of the material from which they were emitted.

### BRIEF DESCRIPTION OF THE DRAWINGS

The following detailed description of the preferred embodiments of the present invention can be best understood when read in conjunction with the following drawings, where like structure is indicated with like reference numerals and in which:

FIG. 1 shows a notional engine block for an internal combustion engine that can be made with a material and casting approach according to an aspect of the present invention;

FIG. 2A and 2B show respectively a calculated phase diagram of a new secondary cast aluminum alloy showing phase transformations as a function of Cu content, and a remnant Cu-containing phase a long solution treatment step for a 319 alloy;

FIG. 3 shows a calculated phase diagram of a cast aluminum alloy with 2% Cu showing phase transformations as a function of Mg content;

FIG. 4 shows a calculated phase diagram of a cast aluminum alloy with 0.5% Cu showing phase transformations as a function of Mg content;

FIG. 5 shows the porosity content as measured by image analysis versus the amount of Cu in the alloy;

FIGS. 6A through 6D show macrographs of eutectic growth morphology of Al-13% Si-0.020% Sr alloys with different Mg additions;

FIGS. 7A and 7B show two different magnification micrographs of the fine equiaxed grains of eutectic without branches of dendrites for the alloy of FIGS. 6A through 6D;

FIGS. 8A and 8B show a cross-section view of a shrinkage sample and a comparison of total shrinkage measured in the shrinkage samples between low Zn (0.1%) and high Zn (0.8%) 319 alloy;

FIGS. 9A through 9C show the effect of Zn content on specific heat, density and surface tension respectively of a 319 alloy;

FIG. 10 shows the effect of Zn on shrinkage and core gas defects in a sand casting of a 319 alloy;

FIGS. 11A and 11B show the effect of Zn content on the fluidity of a 319 alloy using spiral fluidity samples and measured fluidity samples lengths as a function of Zn; and

FIG. 12 shows an image analyzer that can be used to quantify constituent materials in a secondary production aluminum alloy according to an aspect of the present invention.

### DETAILED DESCRIPTION OF THE PREFERRED EMBODIMENTS

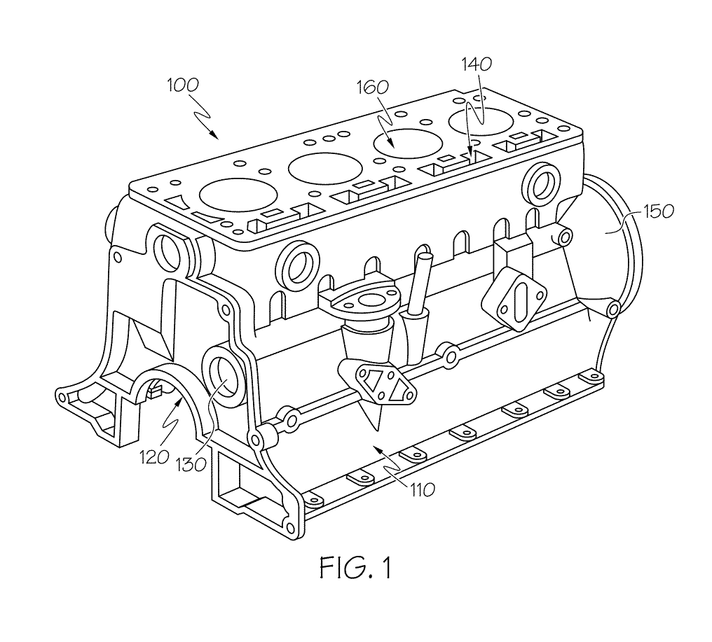

Referring first to FIG. 1, a simplified view of four-cylinder automotive internal combustion engine block 100 is shown. The block 100 includes portions for—among other things—the crankcase 110, the crankshaft bearing 120, the camshaft bearing 130 (in the case of engines with overhead valves and pushrods), water cooling jackets 140, flywheel housing 150 and cylinder bores 160. These bores 160 may include an alloyed surface layer (not shown) that is either integrally formed with the substrate of each bore 160, or as a separate insert or sleeve that is sized to fit securely within. Block 100 is preferably cast from the secondary production aluminum alloy discussed herein, where the alloy is preferably an Al—Si casting alloy (such as alloys 319, 354, 356, 360, 380 and 390). In a preferred form, increases in mechanical properties (such as strengthening, ductility, fatigue resistance or the like) of the block 100 made from the secondary production aluminum alloy raw materials is achieved through post-casting heat treatment. In one particular form, to exhibit the benefits of adding strengthening elements, a casting such as block 100 has to go through optimal solution treatment and aging hardening. Otherwise, the benefit is minimal and it poses adverse influence instead on casting quality.

Improved Alloy Strengthening

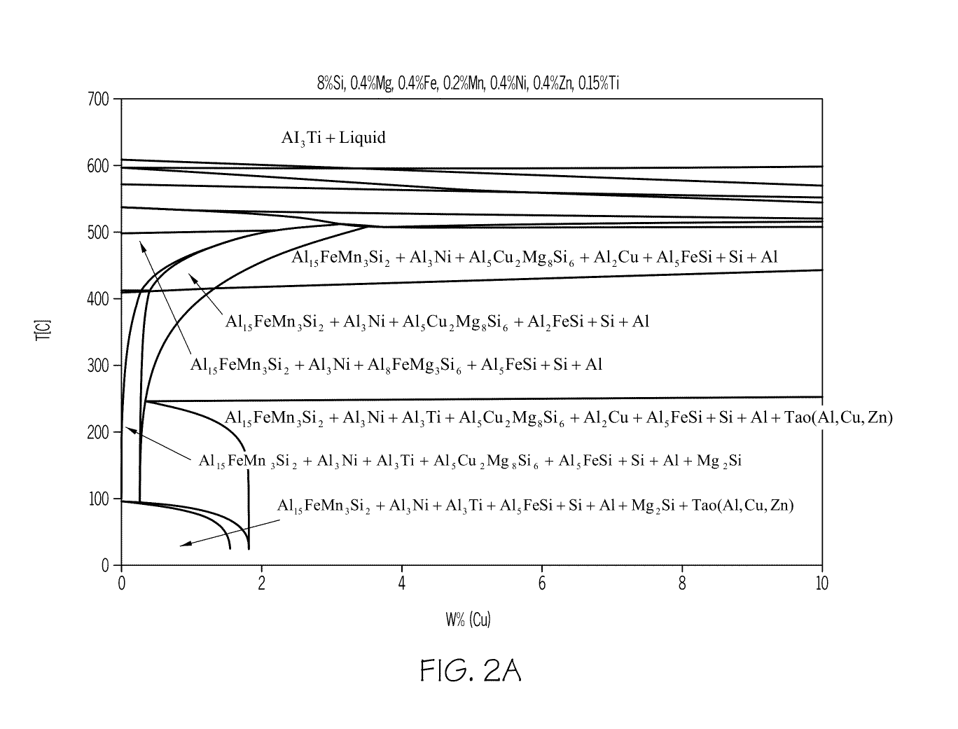

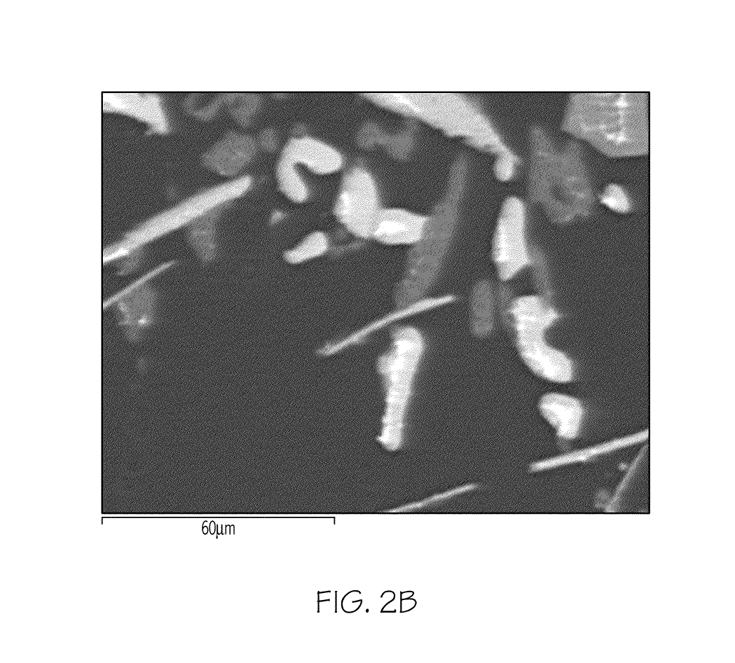

Referring next to FIGS. 2A and 2B and based on calculations from thermodynamic models, particular attention must be paid to castings (such as block 100 of FIG. 1) made from secondary production raw materials that have a high Cu content (for example, 319 or 380 alloys with 3-4% by weight Cu), as they are prone to shrinkage and corrosion. In such circumstances, conventional solution treatment temperatures must be kept below about 500° C., often below 490° C., to avoid incipient melting. As a result, not all the Cu present in the alloy is dissolved into the solid solution, even with a very long solution treatment time (for example, up to about 20 hours). As shown with particularity in FIG. 2B, a Cu-containing phase in a 319 alloy remains even after 24 hours of heat treatment at 495° C.

In fact, it may be that only about 1.5 to 2% of the Cu is dissolved in the aluminum solid solution, as the solubility of Cu in the as-cast condition is very low; this value is near zero when the castings are cooled slowly after solidification. Moreover, the incipient melting problem prohibits further increases in solution temperature beyond the numbers mentioned above. Furthermore, a majority of the Cu present is tied up during solidification with Fe and other elements that form intermetallic phases which have no aging responses in situations where the cast component does not undergo a high temperature solution treatment. Therefore, for the castings (such as HPDC-manufactured components) that are subjected to only a T5 aging process, the Cu content should be kept low, preferably below 0.5% so that all the Cu addition remains in the Al solid solution after solidification. Likewise, in situations when the alloys are subjected to full heat treatment (T6 or T7), the Cu content can be increased up to 2% by weight. Furthermore, it is preferable to control the Cu content below 1.5% by weight and even below 1.0% for corrosion resistant applications, as the solution treatment temperature for the Cu-containing secondary alloy is usually below 500° C. The reduced Cu content also significantly reduces the alloy freezing range and thus shrinkage tendency, which is additionally beneficial as will be discussed below. Examples of components that need corrosion-resistant alloys include transmission cases, oil pans, engine covers, wheels, water pumps and oil pumps, as well as engine and engine components for marine use.

As with Cu, Mg acts as a hardening solute by combining with Si to form Mg/Si precipitates such as β″, β′ and equilibrium Mg₂Si phases, where the actual precipitate type, amount, and sizes depend on aging conditions. Underaging tends to form shearable β″ precipitates, while in peak and overaging conditions unshearable β′ and equilibrium Mg₂Si phases form. Cu can combine with Al, Si and Mg to form many metastable precipitate phases, such as θ′-AlCu, θ-AlCu, and Q-AlSiMgCu. Similar to Mg/Si precipitates, the actual type, size and amount of Cu-containing precipitates depend on aging conditions and alloy compositions. In aluminum alloys, the strengthening due to Cu or Mg precipitates is superior to that of Si alone.

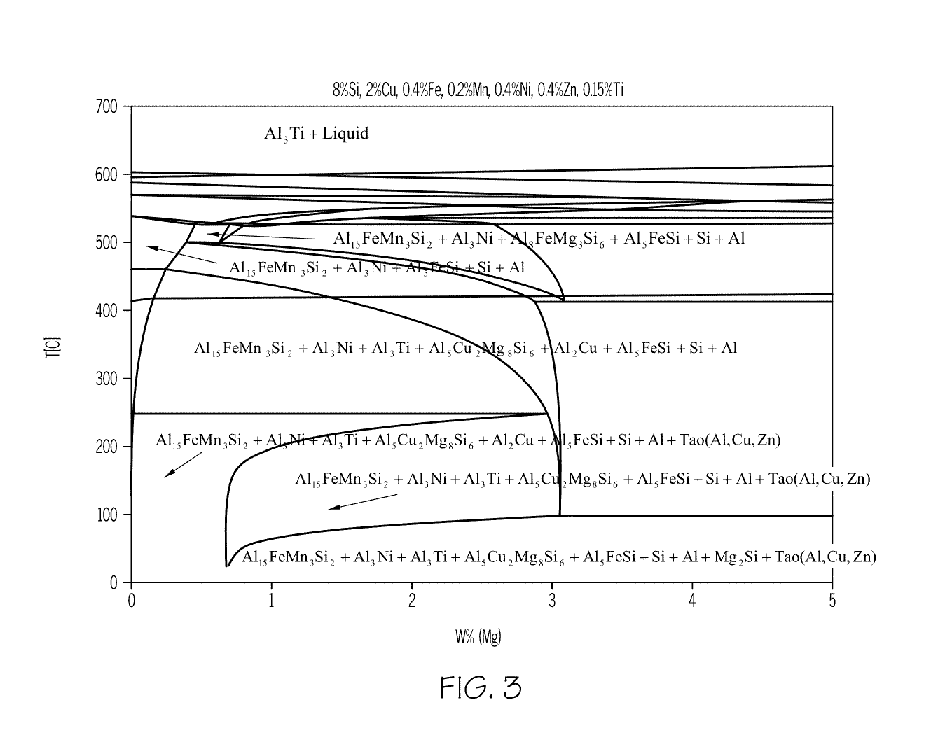

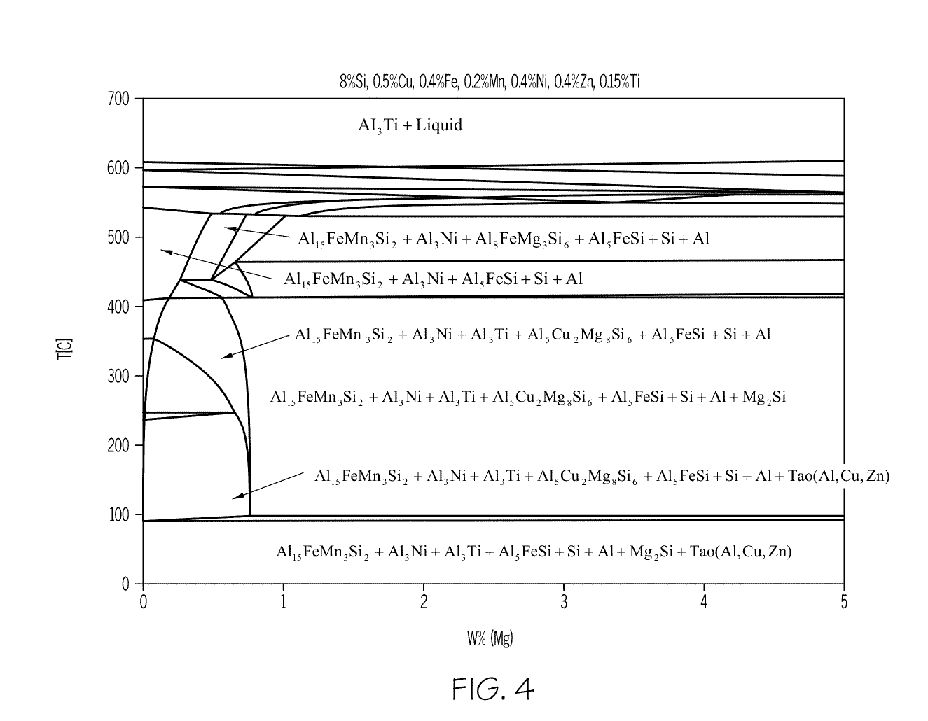

Although Mg is a very effective strengthening element in Al—Si alloy for structural applications below 200° C., preferably below 150° C., its benefit will not show up until the casting is subjected to proper solution treatment and age hardening. Referring next to FIGS. 3 and 4, similar to Cu, the Mg solubility in as-cast Al matrix is also very low, particularly when the casting is cooled very slowly during solidification, such as that which occurs during sand casting. As a result, no strengthening/hardening due to Mg/Si precipitates would be expected without solution heat treatment. As with Cu, for castings subjected to only the T5 aging process, the Mg content should be kept low, in this case, below 0.2%, while in situations when the castings are subjected to full heat treatment (T6 or T7), the Mg content can be increased up to 0.55% by weight. Significantly, the optimal Mg addition depends on Cu content in the alloy, as well as the solution treatment cycle to be used. For example, when the Cu content is about 2%, the safe solution treatment temperature is about 500° C. As shown with particularity in FIG. 3, the maximum solubility of Mg at 500° C. is about 0.35%. It is also noted that the π-Al₈FeMg₃Si₆ phase starts to form when the Mg content is above 0.4%. When the Cu content is reduced to 0.5%, the safe solution treatment temperature can be as high as 520° C., or even 530° C., thus enabling the maximum solubility of Mg to be increased to 0.5%, as shown with particularity in FIG. 4. When Mg is increased above 0.5%, a significant amount of Al₈FeMg₃Si₅ forms, which is difficult to dissolve even with a higher solution treatment at 540° C. for long periods of time, such as 50 hours.

Improved Alloy Castability

In addition to the previously-discussed alloy strengthening improvements, the addition of Cu significantly decreases the melting point and eutectic temperature of the alloy. Therefore, Cu addition increases the solidification freezing range of the alloy, and facilitates the condition of porosity formation. The sequence of solidification and the formation of Cu-rich phases in an Al—Si—Cu—Mg secondary production casting alloy during solidification can be described as follows:

(i) Formation of a primary α-aluminum dendritic network at temperatures below 610° C., leading to a monotonic increase in the concentration of Si and Cu in the remaining liquid.

(ii) At about 560° C. (the Al—Si eutectic temperature), the eutectic mixture of Si and α-Al forms, leading to further increase in Cu content in the remaining liquid.

(iii) At about 540° C., Mg₂Si and Al₈Mg₃FeSi₆ form. When the Cu content is greater than 1.5%, however, the Mg₂Si phase will not form for the alloy containing 0.4% Mg by weight (this is shown in FIG. 2).

(iv) At about 525° C., the eutectic (sometimes called “massive” or “blocky”) CuAl₂ phase forms together with β-Al₅FeSi platelets in the interdendritic regions.

(v) At about 507° C., a eutectic of CuAl₂ with interspersed α-Al forms. In the presence of Mg, the Q phase (Al₅Mg₈Cu₂Si₆) also forms at this temperature, usually with an ultrafine eutectic structure. The tendency to form the blocky CuAl₂ phase is increased by the presence of Sr.

The Cu-free alloy (such as A356) solidifies over a relatively narrow temperature range of about 60° C. and contains nearly 50% of eutectic liquid. Thus, the feeding of the last eutectic liquid to solidify is relatively easy and the level of porosity is normally very low. In the case of an alloy containing Cu (such as 319 and A380), the Cu extends the solidification freezing range to about 105° C. and the fraction of binary eutectic is considerably less than in the Cu-free alloy, thus making the formation of shrinkage porosity much more likely.

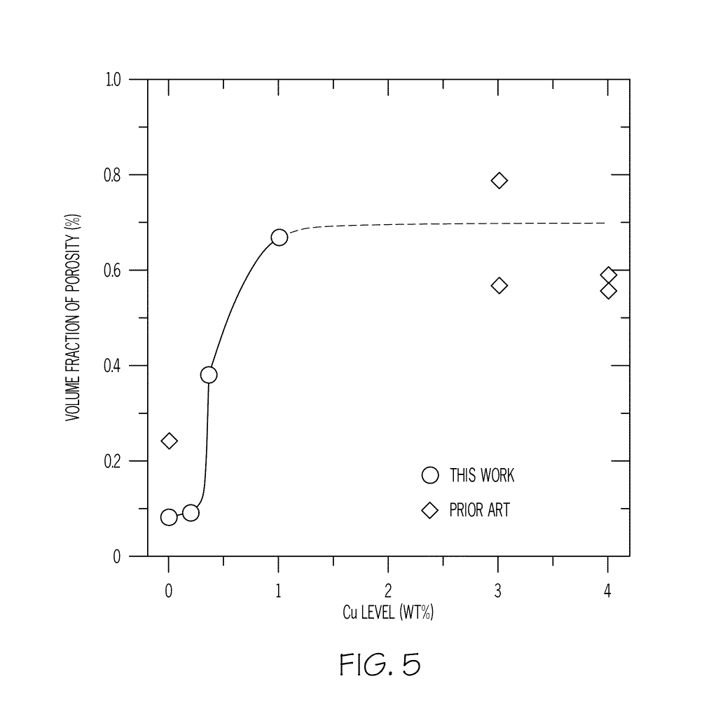

Referring next to FIG. 5, the porosity content (as measured with image analysis) for the different Cu levels is shown. Significantly, the influence of Cu content on microporosity in certain alloys (for example, a Sr-modified Al-7% Si—Cu-0.4% Mg alloy) shows that a dramatic increase in the porosity content occurs when the Cu level increases beyond 0.2%, while the porosity content at a Cu level of 1% is similar to that measured at comparable dendrite arm spacing (DAS) in alloys with 3 and 4% Cu, suggesting that porosity tends to saturate at Cu levels above 1%. As such, it is important to determine what the Cu content of the secondary production aluminum alloy is so that changes to the raw material to control the Cu content to below 1% by weight, and more preferably below 0.5% in order to minimize the detrimental influence of Cu on the tendency of the alloy to shrink.

As with Cu, Si confers several advantages to cast aluminum alloys, most of which applies irrespective of modification. The first and perhaps most important benefit of silicon is that it reduces the amount of shrinkage associated with the freezing of the melt. This is because solid silicon, with its non-close-packed crystal structure, is less dense than the Al—Si liquid solution from which it precipitates. It is generally accepted that shrinkage decreases almost in direct proportion to the silicon content, reaching zero at 25% Si. It is the shrinkage of the eutectic that is important for the castability of hypoeutectic alloys, since the Si in solid solution actually increases the density of the primary α-Al dendrites and therefore slightly increases shrinkage. The shrinkage of the α-Al is about 7%, but this occurs while feeding is easy; the eutectic solidifies in the later stage, when feeding is more difficult, and is reported to have a shrinkage of about 4%. With regard to shrinkage defects, the eutectic alloy is more castable than the hypoeutectic alloy.

The second benefit associated with Si relates to its high latent heat of fusion. It is generally accepted that Si causes an increase in the latent heat of fusion in cast aluminum alloys. The higher latent heats from Si addition mean that the time-to-freezing is extended and this improves fluidity as measured by, for example, spiral fluidity test. It has been observed that the fluidity reaches a maximum in the range 14-16% Si.

Feeding is encouraged by a planar solidification front. Thus, feeding should be easier for pure metals or for eutectics than for alloys with a wide freezing range and an associated mushy zone. From the spiral fluidity test, it was found that the fluidity of Al—Si based alloys reaches the highest near the eutectic composition. This is caused by two associated effects. First, Si content appears to affect the α-Al dendrite morphology, with high Si levels favoring rosettes and lower levels favoring classical α-Al dendrites. In general, rosette-shaped α-Al dendrites make feeding easier by delaying dendrite coherency and reducing the fraction of liquid trapped between the dendrite arms. Mold filling is more difficult in high-cooling rate processes such as permanent mold casting and HPDC because the time-to-freezing is decreased. However, fluidity is increased as the composition approaches the eutectic. As a result, it is preferable to control the Si content in the range of 5-9% for sand and investment castings (which have inherently low cooling rates), 7-10% for permanent metal mold casting and 8-14% for HPDC (which tend to have much higher cooling rates).

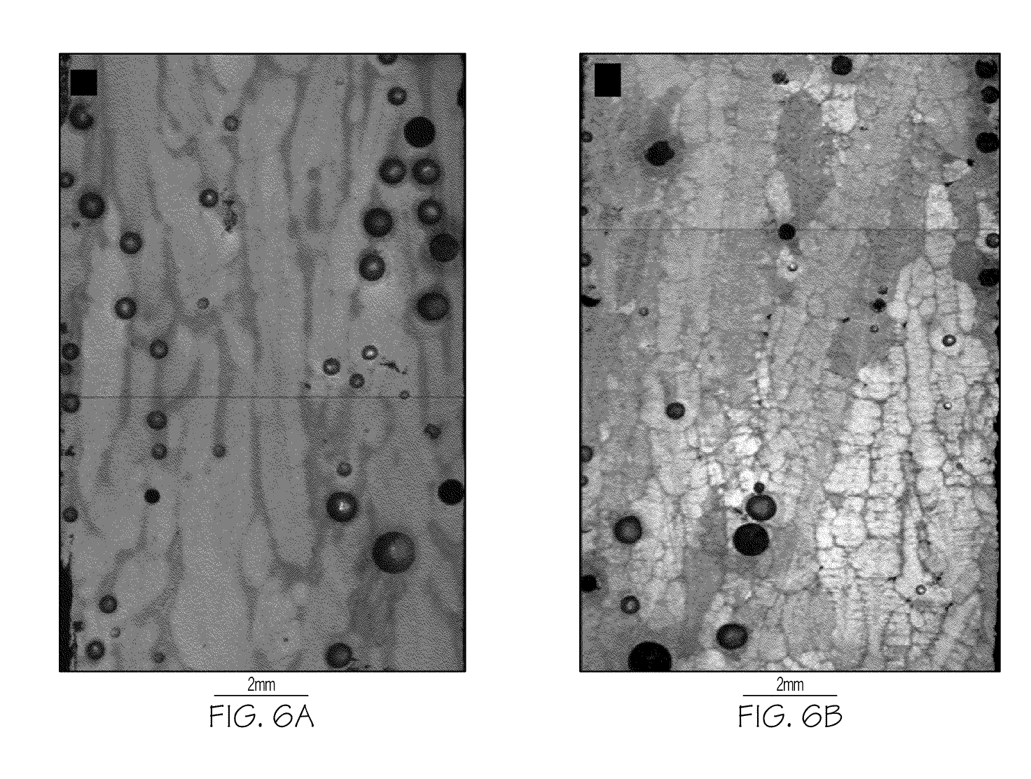

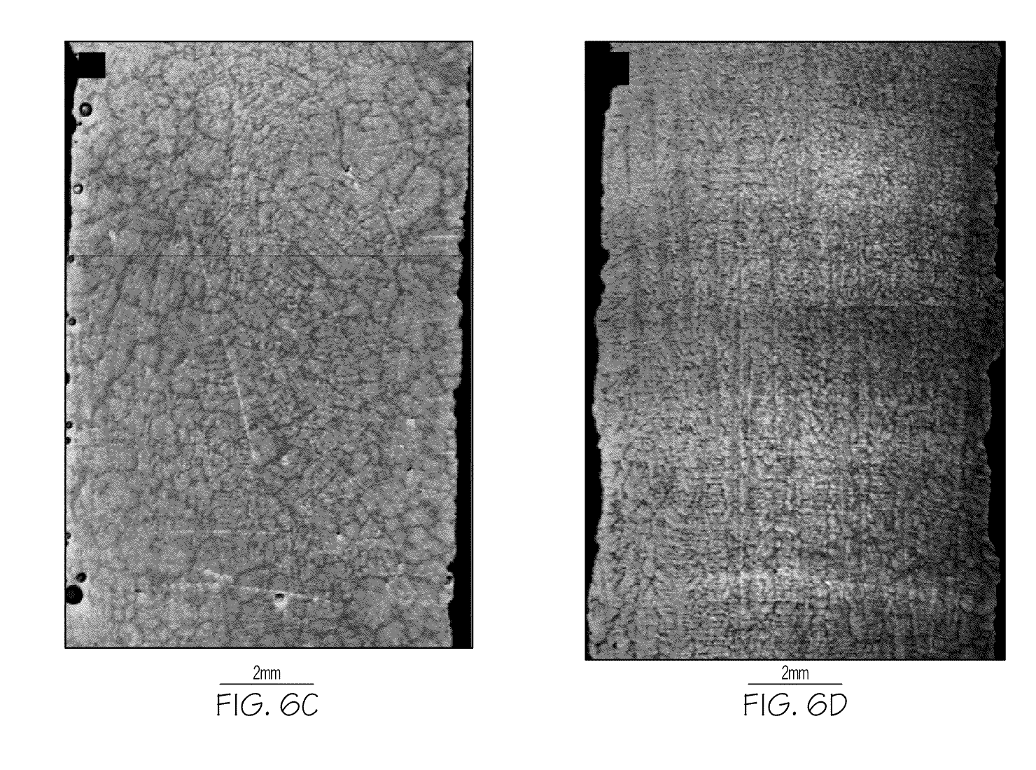

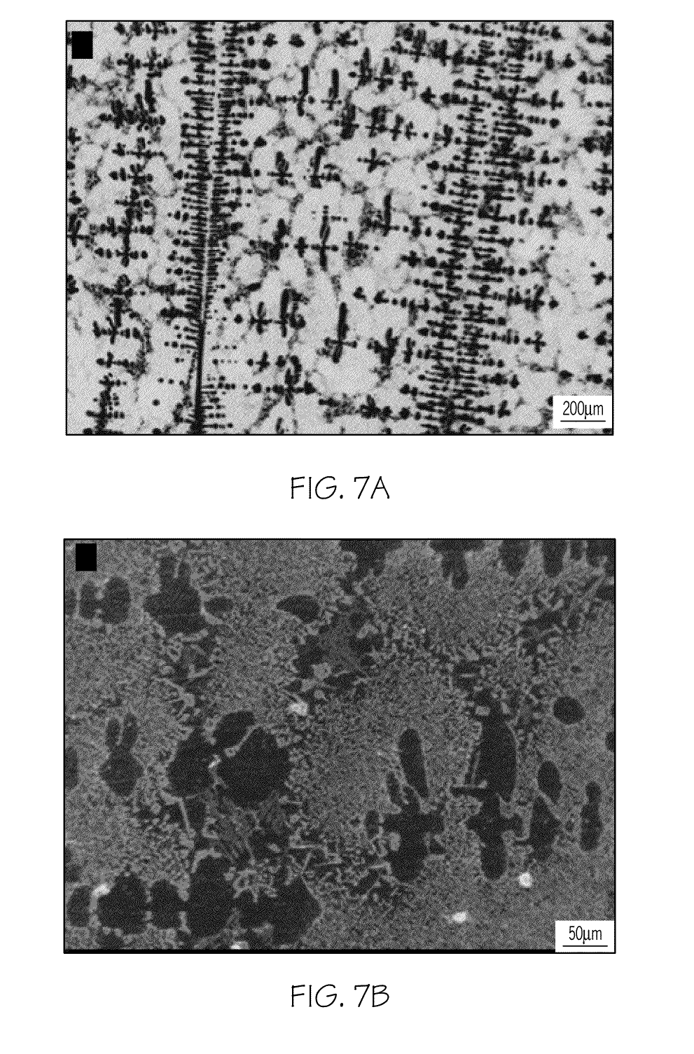

As mentioned in the previous section, the addition of Mg is to increase the tensile strength in cast Al—Si based alloys. Despite this, when the Mg content is increased from 0.4% (such as that in A356) to 0.7% (such as that in A357), the ductility is significantly decreased, particularly in situations where the modified alloy includes Sr. The adverse effect of Mg addition on the ductility is a result of a combination of the higher matrix strength and particularly the increased size and amount of the Fe-rich π-Al₈FeMg₃Si₆intermetallics. Mg addition has also been found affecting Al+Si eutectic structure. Referring next to FIGS. 6A through 6D, macrographs of Al-13% Si-0.020% Sr alloys with different additions of Mg under steady state solidification with a temperature gradient of about 2.1° C./mm and a growth velocity of 0.1 mm/s are shown. For the alloy without the addition of Mg (Mg=0%, G_{L}=2.10° C./mm, R=0.1 mm/s), the eutectic growth morphology presents as cellular, as shown in FIG. 6A. The cell spacing is about 1.7 mm. Unlike other single-phase alloys, however, the cellular eutectic grain boundary is not so straight and contrarily it has small branches that are considered to be related to the interaction with gas bubbles formed in the specimens. Referring with particularity to FIG. 6B, when 0.35% Mg (Mg=0.35%, G_{L}=2.12° C./mm, R=0.1 mm/s) is added into the alloy, columnar eutectic grains are formed, these possess notorious lateral branches although they are not well developed. The primary dendrite cell size of eutectic grains is about 1.8 mm. Referring with particularity to FIG. 6C, when addition of Mg is up to 0.40% (Mg=0.45%, G_{L}=2.13° C./mm, R=0.1 mm/s), the eutectic grains become equiaxed dendrites with an average grain size of 0.8 mm. Interestingly, the microporosity level is significantly reduced except for the edge of the specimen. Referring with particularity to FIG. 6D, when 0.6 wt % of Mg (Mg=0.60%, G_{L}=2.08° C./mm, R=0.1 mm/s) is added to the alloy, a directional grain structure feature can be observed, which is believed to be a result of twinned columnar dendrites of primary α-Al phase with a growth direction approximately opposite to the heat flow as shown in the micrographs of FIGS. 7A and 7B for the Al-13% Si-0.020% Sr alloy (Mg=0.60%, G_{L}=2.08° C./mm, R=0.1 mm/s), showing fine equiaxed grains of eutectic without branches of dendrites. Moreover, the solidified specimen is almost free of microporosity. Of more interest, the eutectic structure includes a large amount of small globular grains with various sizes, of an average size of 0.1 mm. These small equiaxed eutectic grains have no branches, indicating that a great number of heterogeneous sites for eutectic nucleation had operated. From this, the present inventors determined that during solidification of the alloy of FIG. 6D, primary dendrites of α-Al phase first grow protruding into the liquid, after which a great number of eutectic grains nucleate continuously to form fine equiaxed eutectic grains or cells. From the above results based on experiments conducted by the inventors, they have concluded that the addition of Mg considerably alters the nucleation and growth of the eutectic at the same solidification conditions. This Mg impact on the microstructure is valuable in that it provides evidence of casting quality, particularly as it relates to porosity levels.

As indicated above, Fe is a significant impurity in Al alloys, forming brittle complex intermetallics with Al, Si, Mg and other minor constituents. Because these intermetallics seriously degrade the tensile ductility of the alloys and further because they often form during solidification of the eutectic, they affect castability by interfering with inter-dendritic feeding, which in turn leads to the promotion of porosity. The most commonly observed Fe-rich compound is the Al₅FeSi (β-phase), usually found in the Al—Al₅FeSi—Si eutectic as thin platelets interspersed with the silicon flakes or fibers. If Mn is present, the Fe forms Al₁₅(Fe,Mn)₃Si₂ (α-phase), often in the shape of Chinese script. Likewise, if enough Mg is available, the compound Al₈FeMg₃Si₆ (β-phase) is formed, which has a Chinese script appearance if it is formed during the eutectic reaction, or a globular appearance if it forms as a primary precipitate from the liquid. Rapid freezing refines the Fe intermetallics and, thus, the magnitude of the effect of Fe depends on the solidification rate in the casting.

In addition to castability concerns, these Fe-rich intermetallics are usually detrimental to corrosion resistance because they compose a cathode pole (i.e., the inert or noble component of the electrical potential). Compared with other Fe-rich intermetallics such as α-Al₁₅(Fe,Mn)₃Si₂ and π-Al₈FeMg₃Si₆, Al₅FeSi is the more detrimental to corrosion resistance because of its high noble potential. The increased Cu content above 1.5% by weight in the alloy increases the amount of noble Al₂Cu phases facilitating Cu dissolution into α-Al₁₅(Fe,Mn)₃Si₂ This makes potential of the α-Al₁₅(Fe,Mn)₃Si₂ intermetallics even nobler causing a decrease in corrosion resistance.

Reduction and elimination of the β-Al₅FeSi Fe-rich compound can be achieved by controlling the Mn/Fe ratio and the total amount of Mn+Fe. In a preferred form, the Mn/Fe ratio is above 0.5, preferably above 1.0 or higher for most cast components, and to an upper limit of 3.0 or less for components made by HPDC Likewise, the total amount of Mn+Fe should be controlled in a range from 0.4 to 1.0 for minimizing die soldering and the detrimental effect of the Fe-rich intermetallics on material ductility, with a preferable amount between 0.4 to 0.6%.

A high Fe level (up to about 0.8% by weight) may be used for metal mold castings (including HPDC) to avoid hot tearing and die soldering problems, while a lower Fe level (less than 0.5% by weight) should be used for other casting processes. In the presence of Fe, the Mn content may be kept at a level to produce a Mn/Fe ratio greater than 0.3 with a preferable ratio greater than 0.5 as mentioned above.

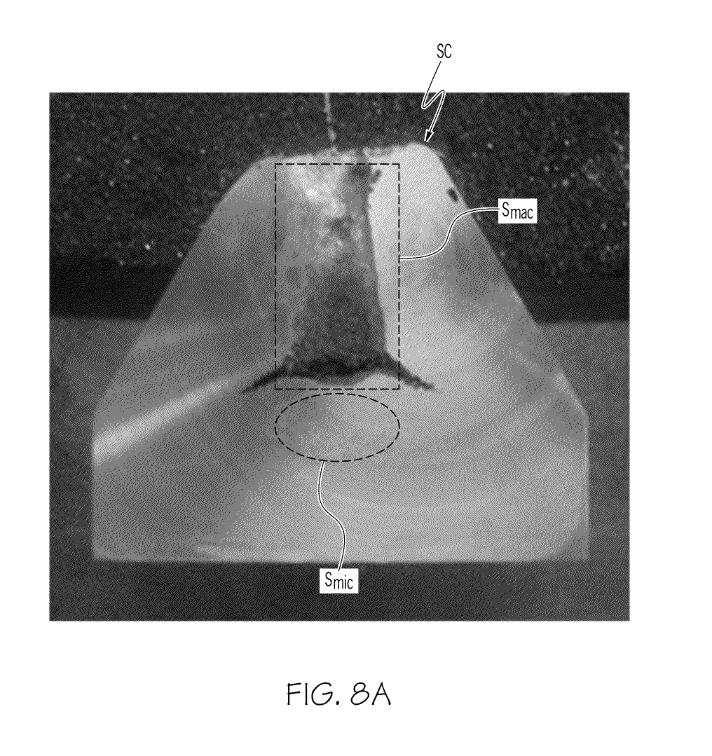

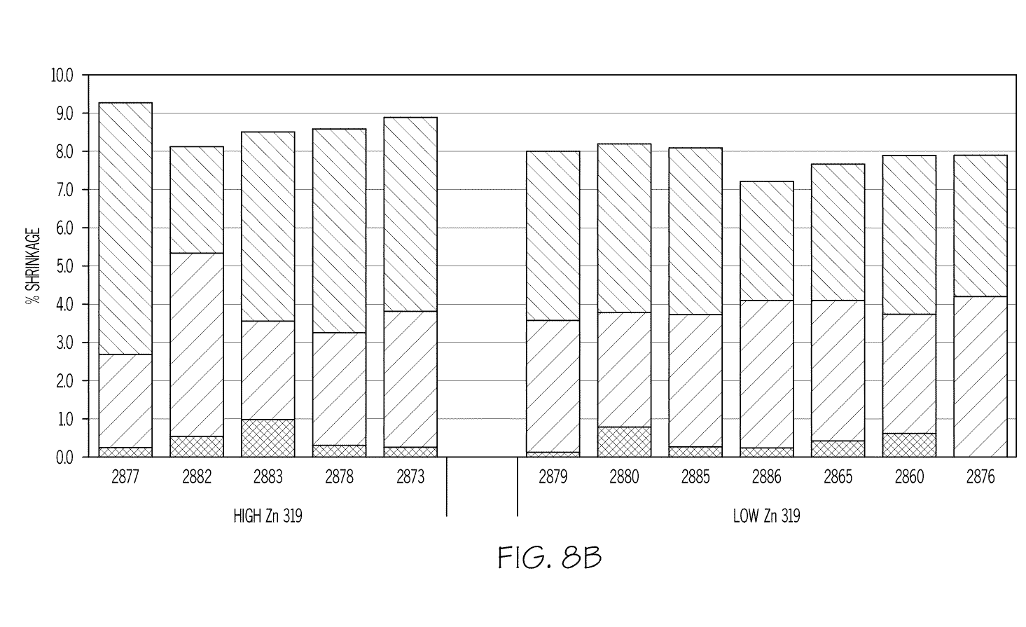

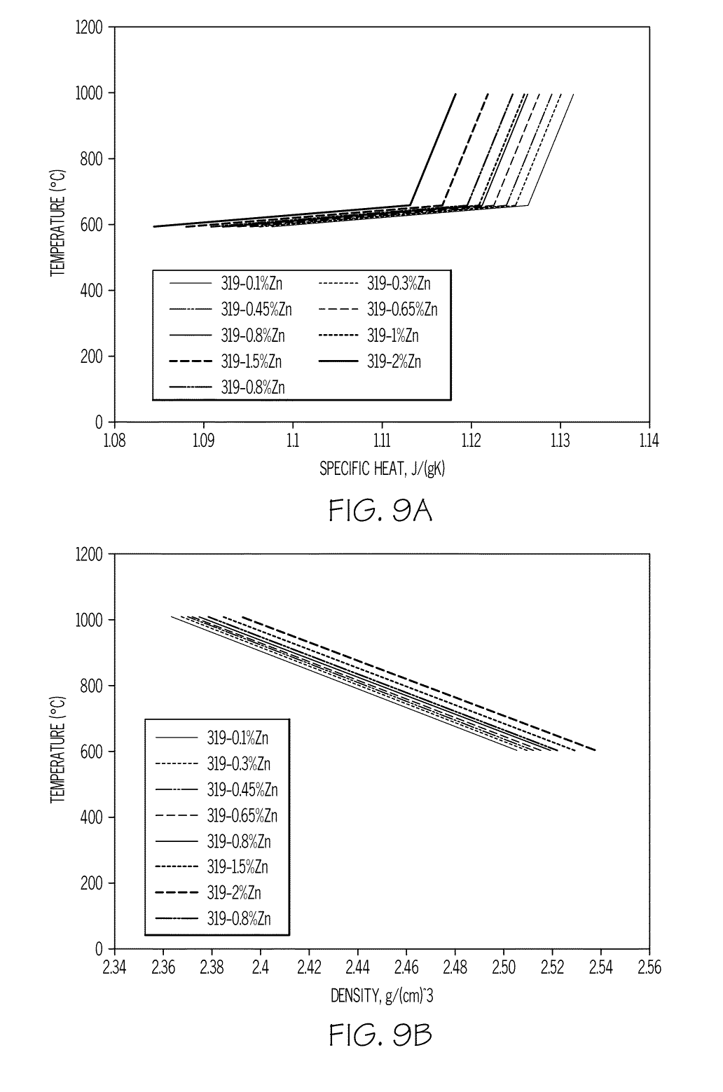

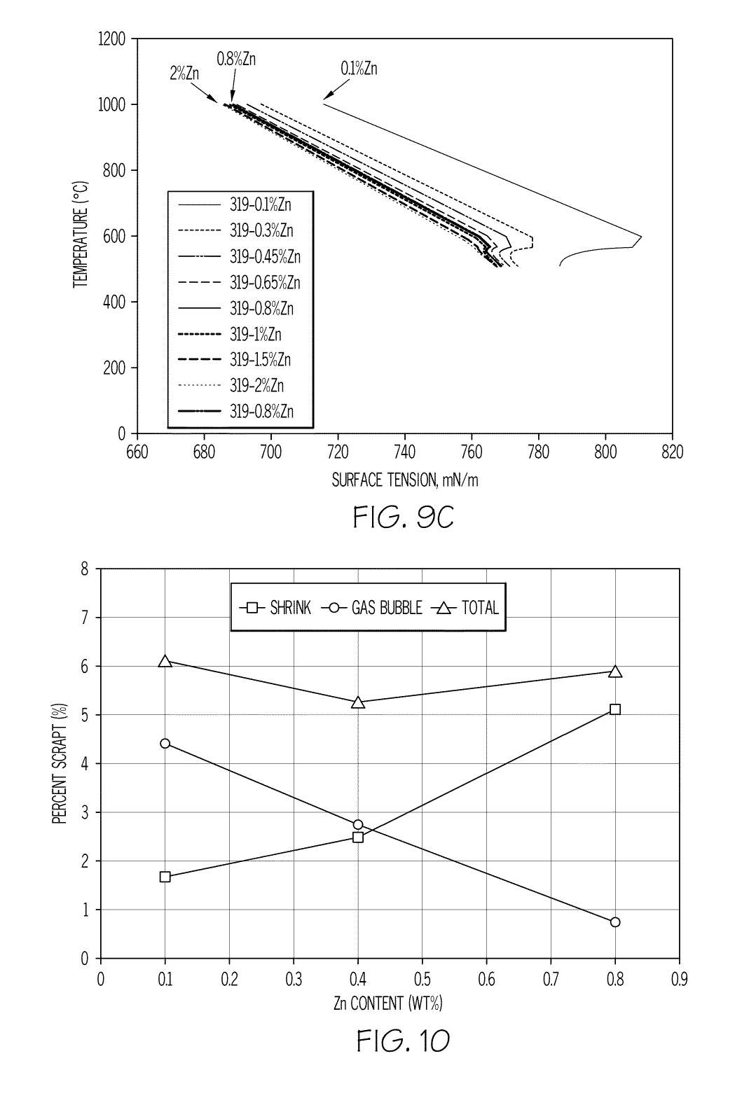

Referring next to FIGS. 8A, 8B through 10, in secondary production aluminum casting alloys in general (and 319 in particular), Zn is present merely as an acceptable impurity element, where the upper limit of Zn is generally thought to be permissible if no more than about 3 wt %. While it is generally accepted that Zn tends to be neutral (i.e., that it neither enhances nor detracts from an alloy's properties), the present inventors believe that Zn affects not only alloy thermal and physical properties but also castability and casting quality. Specifically, the present inventors are of the belief that increasing Zn increases the alloy freezing range and mushy zone size, and thus leads to a tendency to shrink during solidification, as shown by the slumping and contraction SC, macroshrinkage S_{mac} and microshrinkage S_{mic} of the sample pipe in FIGS. 8A and 8B. Increasing Zn also increases alloy density and reduces liquid surface tension and specific heat, as shown in FIGS. 9A through 9C. As a result, the increased Zn not only reduces alloy superficial heat release to a sand core (in the case of sand castings), but also helps expel gas bubbles if they form.

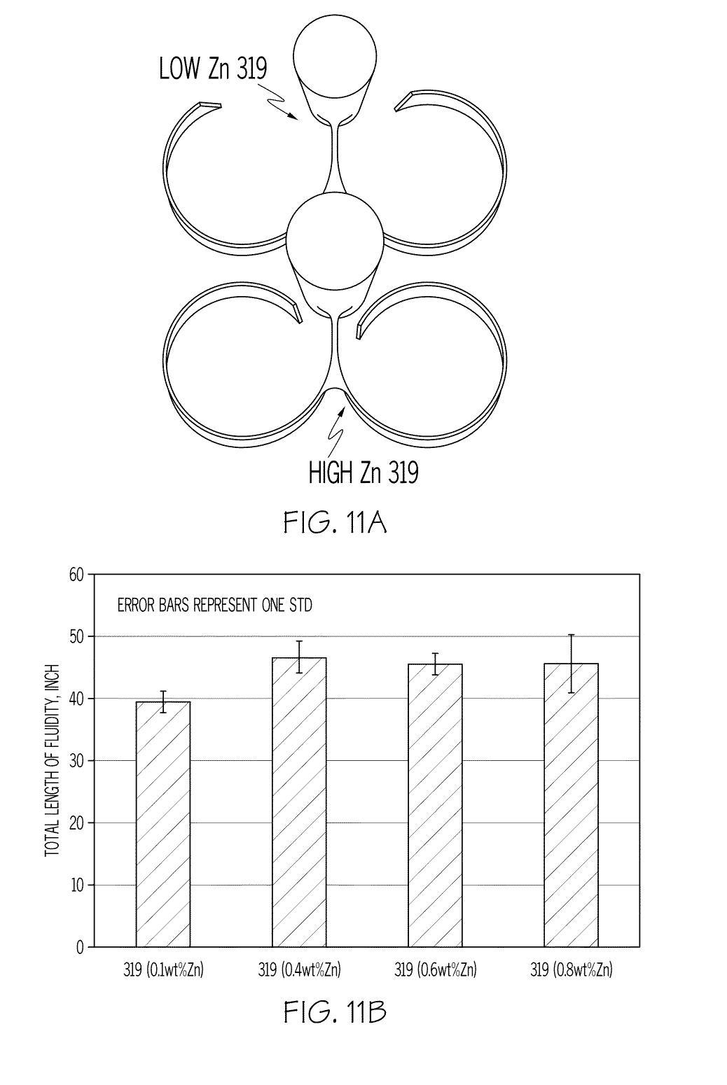

Referring next to FIGS. 11A and 11B, there exists an optimal Zn level (specifically, about 0.4 wt %) at which a good balance between low core gas bubbles and shrink porosity (measured as fluidity) can be achieved. In particular, FIG. 11A shows two spiral fluidity samples tested with two different 319 alloys, one with low Zn and the other with high Zn. In general, the longer spiral equates to a higher fluidity. The higher Zn alloy (which corresponds to the bottom sample in FIG. 11A) shows a longer spiral. Of course, if core gas bubbles are the only concern in production (typically in precision sand casting for engine blocks and semi-permanent mold casting for cylinder heads with chemically bonded sand cores), a high Zn content (specifically, greater than 0.5 but less than 0.8 percent by weight) is recommended. Likewise, when shrinkage is the sole or predominant problem to solve, a low Zn content (less than 0.2 wt %, and preferably less than 0.1 wt %) should be used. When both core gas bubble and shrinkage are present, an optimal Zn level (for example, about 0.4 wt %) should be considered. This logic would also apply to other Al—Si alloys containing Cu and relatively high iron levels (i.e., greater than 0.5%) which are known to be more shrinkage prone. These include aluminum alloys 308, 328, 332, 333, and 339. To facilitate the aging process (such as that used in HPDC where only T5 treatment is generally applied), the Zn concentration should be kept no less than 0.5% by weight. As such, the high fluidity alloy can easily fill the casting with complex shape even with low pouring temperature. This is beneficial in promoting short casting mold fill times, as well as reducing the time for the core gas to penetrate into the liquid metal.

Secondary production cast aluminum alloys may also contain one or more trace elements such as Zr, V, Mo or Co as impurity in the aluminum alloy. The content of the trace elements should be controlled below 0.2% by weight. The present inventors believe that the while the presence of these trace elements in amounts of less than 0.2% can be beneficial for high temperature properties, if the concentration becomes too high, the alloy will lead to undesirably low levels of thermal conductivity, ductility and toughness.

When high Si content (from 7% to 14% and in particular from 10% to 14%) is present in the alloy, Sr should be added to the alloy with a preferable content of 0.01-0.02% by weight for the hypoeutectic alloy (i.e., less than 12% Si) and 0.04-0.05% by weight for the hypereutectic alloy (i.e., greater than 12% Si). The modified Si morphology can improve the ductility and fracture toughness of the raw material. It is also recommended to refine both primary aluminum dendrite grains and the eutectic (Al—Si) grains to improve the castability and corrosion resistance. To do so, the Ti and B contents in the alloy should be kept above 0.15% and 0.005% by weight, respectively for the hypoeutectic, while the Sr and B contents should be controlled at about 0.04 to about 0.05%, and about 0.025% to about 0.03%, respectively for the near-eutectic alloys where there is about 12-14% Si.

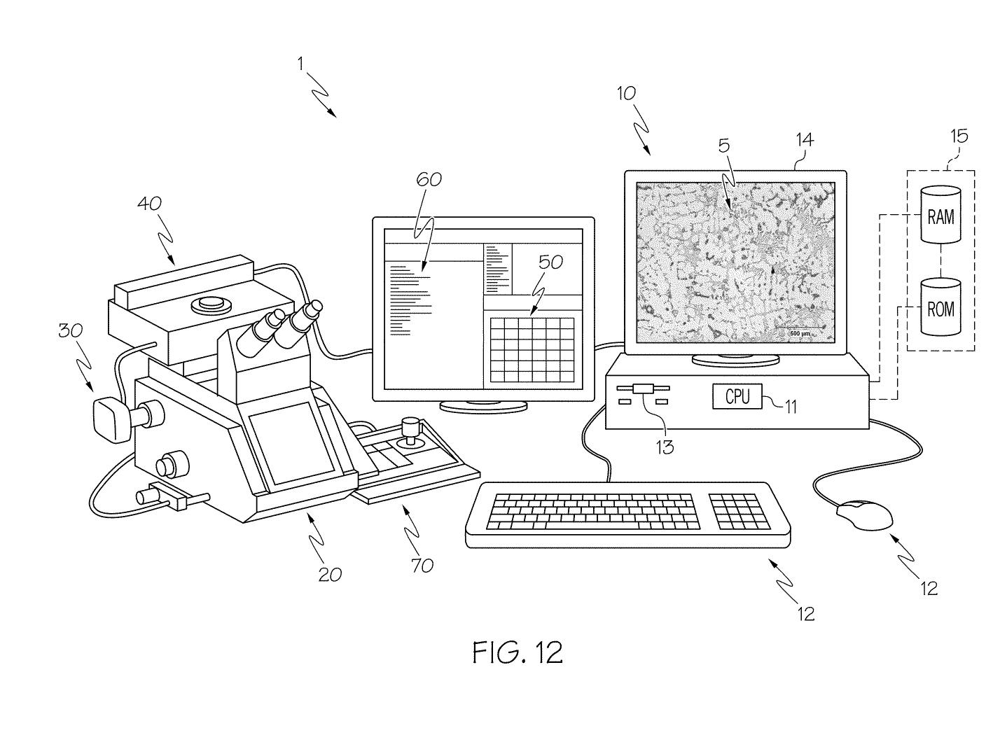

Significantly, the production of secondary aluminum will need to take advantage of frequent measuring or analysis (such as by chemistry analysis—such as the ICP mentioned above—and image analysis) of the alloy composition during the various recycling, melting, casting and post-casting steps to determine if the concentration of the alloy strengthening ingredients (such as the aforementioned Cu and Mg), the alloy castability ingredients (such as the aforementioned Cu, Si, Mg, Fe, Mn, Zr and trace others such as Zr, V, Mo and Co) and the eutectic grain modifiers (such as the aforementioned Sr) is within predetermined tolerances based on the component being fabricated. Furthermore, it may be advantageous to overheat the liquid material that is created from the secondary production raw material (for example, up to 1000° C. for 15 to 30 minutes as mentioned above) Likewise, to the extent that one or more of these elements or related ingredients may contaminate the alloy, it is important to analyze samples of secondary production materials to determine if these tight tolerances are being maintained. In one form, an image analyzer (also referred to as an image analysis system, as shown in FIG. 12 may be used to ensure that the secondary production aluminum alloys are within predetermined constituent compositions in the manner commensurate with the design needs of the component being cast from such material. The image analyzer is in the form of a computerized vision system 1 that is configured to perform data gathering, analysis and manipulation necessary to quantify material constituents, microstructures or the like. System 1 includes a computer 10 or related data processing equipment that includes a processing unit 11 (which may be in the form of one or more microprocessors or related processing means), one or more mechanisms for information input 12 (including a keyboard, mouse or other device, such as a voice-recognition receiver (not shown)), as well as a one or more loaders 13 (which may be in the form of magnetic or optical memory or related storage in the form of CDs, DVDs, USB port or the like), one or more display screens or related information output 14, a memory 15 and computer-readable program code means (not shown) to process at least a portion of the received information relating to the aluminum alloy. As will be appreciated by those skilled in the art, memory 15 may be in the form of random-access memory (RAM, also called mass memory, which can be used for the temporary storage of data) and instruction-storing memory in the form of read-only memory (ROM). In addition to other forms of input not shown (such as through an internet or related connection to an outside source of data), the loaders 13 may serve as a way to load data or program instructions from one computer-usable medium (such as flash drives or the aforementioned CDs, DVDs or related media) to another (such as memory 15). As will be appreciated by those skilled in the art, computer 10 may exist as an autonomous (i.e., stand-alone) unit, or may be the part of a larger network such as those encountered in cloud computing, where various computation, software, data access and storage services may reside in disparate physical locations. Such a dissociation of the computational resources does not detract from such a system being categorized as a computer.

In a particular form, the computer-readable program code that contains algorithms and formulae needed to analyze alloy constituents can be loaded into ROM that is part of memory 15. Such computer-readable program code may also be formed as part of an article of manufacture such that the instructions contained in the code are situated on a magnetically-readable or optically-readable disk or other related non-transitory, machine-readable medium, such as flash memory device, CDs, DVDs, EEPROMs, floppy disks or other such medium capable of storing machine-executable instructions and data structures. Such a medium is capable of being accessed by computer 10 or other electronic device having processing unit 11 used for interpreting instructions from the computer-readable program code. As will be understood by those skilled in the computer art, a computer 10 that forms a part of image analysis system 1 may additionally include additional chipsets, as well as a bus and related wiring for conveying data and related information between processing unit 11 and other devices (such as the aforementioned input, output and memory devices). Upon having the program code means loaded into ROM, the computer 10 of system 1 becomes a specific-purpose machine configured to determine the elemental makeup of a cast component in the manner as described herein. In another aspect, system 1 may be just the instruction code (including that of the various program modules (not shown)), while in still another aspect, system 1 may include both the instruction code and a computer-readable medium such as mentioned above.

It will also be appreciated by those skilled in the art that there are other ways to receive data and related information besides the manual input approach depicted in input 12 (especially in situations where large amounts of data are being input), and that any conventional means for providing such data in order to allow processing unit 11 to operate on it is within the scope of the present invention. As such, input 12 may also be in the form of high-throughput data line (including the internet connection mentioned above) in order to accept large amounts of code, input data or other information into memory 15. The information output 14 is configured to convey information relating to the desired casting approach to a user (when, for example, the information output 14 is in the form of a screen as shown) or to another program or model. It will likewise be appreciated by those skilled in the art that the features associated with the input 12 and output 14 may be combined into a single functional unit such as a graphical user interface (GUI).

The IA system 1 is used to extract information from images 5, in particular, using metallographic techniques to acquire images of the casting sample or material specimen of interest. Starting with a prepared (for example, polished) metallographic sample, a microscope 20 or related scanner or visual acquisition device is used to magnify and display on output 14 the image 5 that is captured by the camera 30. Typically, many images 5 are captured through the use of a motorized stage 40 and stage pattern 50. Gray thresholding may then be performed on these digitized images 5 in a computer-based routine or algorithm 60 (shown in user-readable form on a display) that make up the image analysis software that may be stored in memory 15 or other suitable computer-readable medium. A stage controller 70 (which employs joy stick-like control) may be used to move the micrograph of the material sample from one field to another field in the microscope 20 through a three-dimensional (Cartesian) series of x, y and z (focus) stage movements. This allows movement across a stage pattern 50 to permit analyzing multiple fields of view over the sample. This automated stage pattern 50—which includes auto focus features—permits the capture of large amounts of data in a short period of time. The joy stick of stage controller 70 allows movement of the stage while observing the sample through the eyepiece of microscope 20 to facilitate the selection of particular areas that the analysis of the present invention will be performed on.

In addition to analysis, the production of secondary aluminum will need to take advantage of making as-needed alloying composition additions or adjustments during melting or recycling steps, depending on the intended end-use of the alloy being produced. Additional adjustments may be made by adding primary recycle alloys ingredients or pre-made master alloys. In one form, constituent information gleaned from the IA system 1 may be used to determine which additives (and in which quantity) will need to be included in the alloy casting or related preparation steps.

At least in a production-based environment, the present inventors believe that a spectrometer with ICP is a preferred way to analyze the compositions, and this would be particularly beneficial in situations where secondary production aluminum alloys are being used, as the normal raw material quality controls present in primary production alloys may not be available or as sensitive. This approach is particularly well-suited to identifying ingredient metals that are present in extremely low concentrations. In one form, concentrations as low as one part per quadrillion may be identified with ICP.

It is noted that terms like “preferably”, “commonly” and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention. Likewise, terms such as “substantially” are utilized to represent the inherent degree of uncertainty that may be attributed to any quantitative comparison, value, measurement, or other representation. It is also utilized to represent the degree by which a quantitative representation may vary from a stated reference without resulting in a change in the basic function of the subject matter at issue.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. An aluminum alloy consisting essentially of raw materials by weight approximately 5 to 14% silicon, 0 to 1.5% copper, 0.2 to 0.55% magnesium, 0.2 to 1.2% iron, 0.1 to 0.6% manganese, 0 to 0.5% nickel, 0 to 0.8% zinc, 0 to 0.2% of other trace elements selected from the group consisting essentially of titanium, zirconium, vanadium, molybdenum and cobalt, and the balance aluminum, wherein at least a portion of said balance aluminum comprises secondary production aluminum.

2. The aluminum alloy of claim 1, wherein at least a majority of said balance aluminum comprises secondary production aluminum.

3. The aluminum alloy of claim 1, wherein a substantial entirety of said balance aluminum comprises secondary production aluminum.

4. The aluminum alloy of claim 1, wherein said silicon is by weight approximately 5 to 8%, said copper is by weight approximately 0 to 1.0%, said magnesium is by weight approximately 0.2 to 0.4%, said iron is by weight no more than approximately 0.4%, said manganese is by weight approximately 0 to 0.2%, said nickel is by weight approximately 0 to 0.2% and said zinc is by weight approximately 0 to 0.3%.

5. The aluminum alloy of claim 1, wherein said silicon is by weight approximately 8 to 14%, said copper is by weight approximately 1.0 to 1.5%, said magnesium is by weight approximately 0.4 to 0.55%, said iron is by weight no more than approximately 0.8%, said manganese is by weight approximately 0 to 0.3%, said nickel is by weight approximately 0 to 0.5% and said zinc is by weight approximately 0 to 0.5%.

6. The aluminum alloy of claim 1, wherein said copper and said magnesium by weight are below approximately 0.5% and 0.2%, respectively.

7. A method of forming a cast automotive component, said method comprising:
heating a quantity of raw materials at least a portion of which comprises secondary production raw materials until at least a substantial majority thereof melts to become by weight approximately 5 to 14% silicon, 0 to 1.5% copper, 0.2 to 0.55% magnesium, 0.2 to 1.2% iron, 0.1 to 0.6% manganese, 0 to 0.5% nickel, 0 to 0.8% zinc, 0 to 0.2% of other trace elements selected from the group consisting essentially of titanium, zirconium, vanadium, molybdenum and cobalt, and the balance aluminum;
placing said heated quantity of raw material into a mold that substantially defines the shape of said component; and
cooling said melted quantity of raw materials.

8. The method of claim 7, further comprising:
determining whether the presence of at least one alloying ingredient is within tolerance;
and adjusting an amount of at least one of said alloying ingredients that is outside said tolerance.

9. The method of claim 7, wherein said component is selected from the group consisting of an engine block and cylinder head.

10. The method of claim 9, wherein said aluminum alloy comprises at least one of a high ductility alloy or a high fatigue strength alloy wherein said silicon is by weight approximately 5 to 8%, said copper is by weight approximately 0 to 1.0%, said magnesium is by weight approximately 0.2 to 0.4%, said iron is by weight no more than approximately 0.4%, said manganese is by weight approximately 0 to 0.2%, said nickel is by weight approximately 0 to 0.2% and said zinc is by weight approximately 0 to 0.3%.

11. The method of claim 9, wherein said aluminum alloy comprises a high tensile strength alloy wherein said silicon is by weight approximately 8 to 14%, said copper is by weight approximately 1.0 to 1.5%, said magnesium is by weight approximately 0.4 to 0.55%, said iron is by weight no more than approximately 0.8%, said manganese is by weight approximately 0 to 0.3%, said nickel is by weight approximately 0 to 0.5% and said zinc is by weight approximately 0 to 0.5%.

12. The method of claim 9, wherein said aluminum alloy comprises a high pressure die cast alloy wherein said copper and said magnesium by weight are below approximately 0.5% and 0.2%, respectively.

13. The method of claim 7, wherein at least a majority of said balance aluminum comprises secondary production aluminum.

14. The method of claim 7, wherein said mold is selected from the group consisting of a sand mold, lost foam mold, die cast mold, permanent (gravity) mold or combinations thereof.

15. The method of claim 7, wherein said heating takes place in a furnace and said cooling takes place in a mold.

16. The method of claim 7, wherein said heating comprises overheating in order to substantially eliminate any residual atomic cluster that may be present in said heated quantity of raw material.

17. A method of verifying the casting quality of an aluminum alloy, the method comprising:
receiving a sample of said aluminum alloy at least a portion of which comprises secondary production raw materials;
generating a microstructure image corresponding to a location of interest in said sample;
measuring at least one indicia within said image; and
correlating said indicia with the presence of at least alloy constituent or at least one contaminant within said alloy.

18. The method of claim 17, wherein at least one of said receiving, generating and measuring comprises performing a metallographic analysis.

19. The method of claim 18, wherein said metallographic analysis is performed by a microstructure image analysis system with at least one algorithm programmed therein to measure at least one phase fraction.

20. The method of claim 17, wherein said at least a portion of said secondary production raw materials comprises a majority by weight of secondary production aluminum.

21. The method of claim 17, wherein said aluminum alloy consists essentially of by weight approximately 5 to 14% silicon, 0 to 1.5% copper, 0.2 to 0.55% magnesium, 0.2 to 1.2% iron, 0.1 to 0.6% manganese, 0 to 0.5% nickel, 0 to 0.8% zinc, 0 to 0.2% of other trace elements selected from the group consisting essentially of titanium, zirconium, vanadium, molybdenum and cobalt, and the balance aluminum.

22. The method of claim 17, wherein said indicia comprises an iron intermetallic phase volume fraction.
